(19) United States
(12) Patent Application Publication
(10) Pub. No.: US 2010/0114195 A1
(43) Pub. Date: May 6, 2010

(54) IMPLANTABLE MEDICAL DEVICE INCLUDING EXTRAVASCULAR CARDIAC STIMULATION AND NEUROSTIMULATION CAPABILITIES

(75) Inventors: John E. Burnes, Coon Rapids, MN (US); Chris Zillmer, Scandia, MN (US); Paul G. Krause, Shoreview, MN (US); Gerald P. Arne, Long Lake, MN (US); Timothy Davis, Coon Rapids, MN (US); David J. Peichel, Minneapolis, MN (US); James D. Reinke, Maple Grove, MN (US); William T. Donofrio, Andover, MN (US); Xiaohong Zhou, Woodbury, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/362,662

(22) Filed: Jan. 30, 2009

Related U.S. Application Data
(60) Provisional application No. 61/110,124, filed on Oct. 31, 2008.

(51) Int. Cl. A61N 1/39; A61N 1/36; A61N 1/362

(52) U.S. Cl. 607/4; 607/5; 607/9

(57) ABSTRACT

An implantable medical device may deliver pacing, cardioversion, and/or defibrillation stimulation to a heart of a patient via extravascular electrodes and delivers electrical stimulation to a nonmyocardial tissue site to modulate the autonomic nervous system of the patient. The implantable medical device may include a cardiac therapy module that generates and delivers at least one of pacing, cardioversion, or defibrillation therapy to a patient via an extravascular electrode, and a neurostimulation therapy module that generates and delivers a neurostimulation signal to the patient via a neurostimulation electrode. The cardiac therapy module and neurostimulation therapy module may be disposed in a common housing of the medical device. In some examples, at least one common lead may electrically couple the neurostimulation electrode and the extravascular electrode to the neurostimulation and cardiac therapy modules, respectively.

This application claims the benefit of U.S. Provisional Application No. 61/110,124, entitled, “IMPLANTABLE MEDICAL DEVICE INCLUDING EXTRAVASCULAR CARDIAC STIMULATION AND NEUROSTIMULATION CAPABILITIES,” and filed on Oct. 31, 2008, the entire content of which is incorporated herein by reference.

### TECHNICAL FIELD

The disclosure relates to medical devices, and, more particularly, medical devices that deliver electrical stimulation therapy to a patient.

### BACKGROUND

A wide variety of implantable medical devices for delivering a therapy or monitoring a physiologic condition of a patient have been clinically implanted or proposed for clinical implantation in patients. Some implantable medical devices may employ one or more elongated electrical leads and/or sensors. Such implantable medical devices may deliver therapy or monitor the heart, muscle, nerve, brain, stomach or other organs. In some cases, implantable medical devices may deliver electrical stimulation therapy and/or monitor physiological signals via one or more electrodes or sensor elements, which may be included as part of one or more elongated implantable medical leads. Implantable medical leads may be configured to allow electrodes or sensors to be positioned at desired locations for delivery of stimulation or sensing electrical depolarizations. For example, electrodes or sensors may be located at a distal portion of the lead. A proximal portion of the lead may be coupled to an implantable medical device housing, which may contain electronic circuitry such as stimulation generation and/or sensing circuitry.

For example, implantable cardiac therapy devices, such as cardiac pacemakers or implantable cardioverter defibrillators, provide therapeutic stimulation to the heart by delivering electrical therapy signals such as pulses or shocks for pacing, cardioversion or defibrillation pulses via electrodes of one or more implantable leads. In some cases, such an implantable medical device may sense intrinsic depolarizations of the heart, and control the delivery of such signals to the heart based on the sensing. When an abnormal cardiac rhythm is detected, such as bradycardia, tachycardia or fibrillation, an appropriate electrical therapy (e.g., in the form of pulses) may be delivered to restore the normal cardiac rhythm. For example, in some cases, an implantable cardiac therapy device may deliver pacing, cardioversion or defibrillation therapy to the heart of the patient upon detecting ventricular tachycardia, and deliver cardioversion or defibrillation therapy to a patient's heart upon detecting ventricular fibrillation.

### SUMMARY

In general, the disclosure is directed an implantable medical device that includes a cardiac therapy module and a neurostimulation therapy module in a common housing. The cardiac therapy module may provide cardiac stimulation, e.g., pacing, cardioversion, and/or defibrillation to a patient via two or more extravascular electrodes (e.g., subcutaneous electrodes). The neurostimulation module may provide electrical stimulation therapy to a tissue site within a patient, such as a nonmyocardial tissue site (e.g., tissue proximate a nerve) or a nonvascular cardiac tissue site (e.g., a cardiac fat pad). In some examples, the neurostimulation module may provide stimulation to modulate an autonomic nervous system of the patient, which may provide cardiac benefits that complement the cardiac stimulation provided by the cardiac therapy module.

In one aspect, the disclosure is directed to a system comprising a housing, at least two extravascular electrodes implanted within a patient, a cardiac therapy module that delivers at least one of pacing, cardioversion, or defibrillation therapy to the patient via the at least two extravascular electrodes, a neurostimulation electrode, and a neurostimulation therapy module that delivers a neurostimulation signal to the patient via the neurostimulation electrode. The cardiac therapy module and the neurostimulation therapy module are enclosed in the housing.

In another aspect, the disclosure is directed to a method comprising generating at least one of pacing, cardioversion, or defibrillation therapy with a cardiac therapy module in a housing of a medical device, delivering the at least one of pacing, cardioversion, or defibrillation therapy to a patient via at least two extravascular electrodes implanted within a patient, generating a neurostimulation signal with a neurostimulation therapy module in the housing of the medical device, and delivering the neurostimulation signal to the patient via a neurostimulation electrode.

In another aspect, the disclosure is directed to a system comprising means for generating and delivering at least one of pacing, cardioversion, or defibrillation therapy via at least two extravascular electrodes implanted within a patient, means for generating and delivering a neurostimulation signal to the patient via a neurostimulation electrode, and a housing enclosing the means for generating and delivering the at least one of pacing, cardioversion, or defibrillation therapy and means for generating and delivering the neurostimulation signal.

In another aspect, the disclosure is directed to a computer-readable medium comprising instructions. The instructions cause a programmable processor to perform any part of the techniques described herein.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1A is a conceptual diagram illustrating an example therapy system that delivers cardiac and neurostimulation therapy.

FIG. 1B is a conceptual diagram illustrating another example therapy system that delivers cardiac and neurostimulation therapy.

FIG. 2A is a conceptual diagram illustrating the therapy system of FIG. 1B in greater detail.

FIG. 2B is a conceptual diagram illustrating another example therapy system.

FIG. 2C is a conceptual diagram illustrating another example therapy system.

FIG. 3 is a functional block diagram of an example implantable medical device that includes cardiac and neurostimulation modules.

FIG. 4 is block diagram of an example medical device programmer.

FIG. 5 is a flow diagram illustrating an example technique for delivering both cardiac and neurostimulation therapy to a patient.

### DETAILED DESCRIPTION

In general, the disclosure is directed toward therapy systems that include an implantable medical device that delivers pacing, cardioversion, and/or defibrillation stimulation to a heart of a patient via extravascular electrodes and delivers electrical stimulation to a tissue site, such as a nonmyocardial tissue site or a nonvascular cardiac tissue site. The nonmyocardial tissue site may be intravascular and/or extravascular. In some examples, the electrical stimulation may be provided to stimulate the autonomic nervous system of the patient. In examples described herein, an extravascular implantable cardioversion defibrillator (ICD) system includes a cardioversion defibrillation signal generator and a neurostimulation signal generator in a common housing. An extravascular electrode may include, for example, a subcutaneous electrode, a submuscular electrode, an epicardial electrode or an intramural electrode. In some examples, however, an extravascular electrode may not include an electrode that contacts the patient's heart. Thus, in some examples described herein, an extravascular electrode may not include epicardial or intramural electrodes located within the heart.

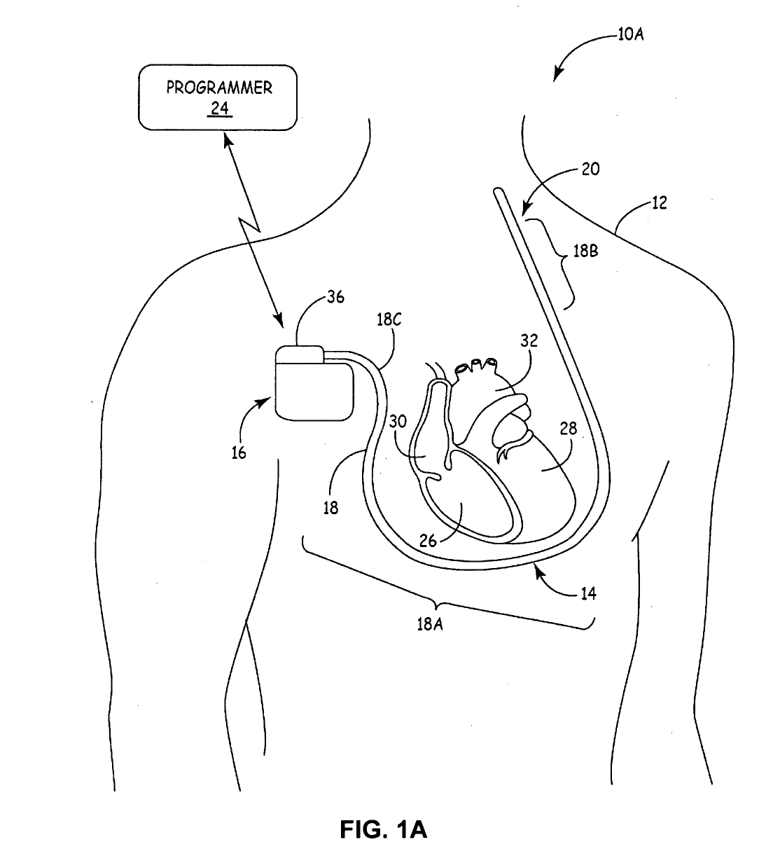

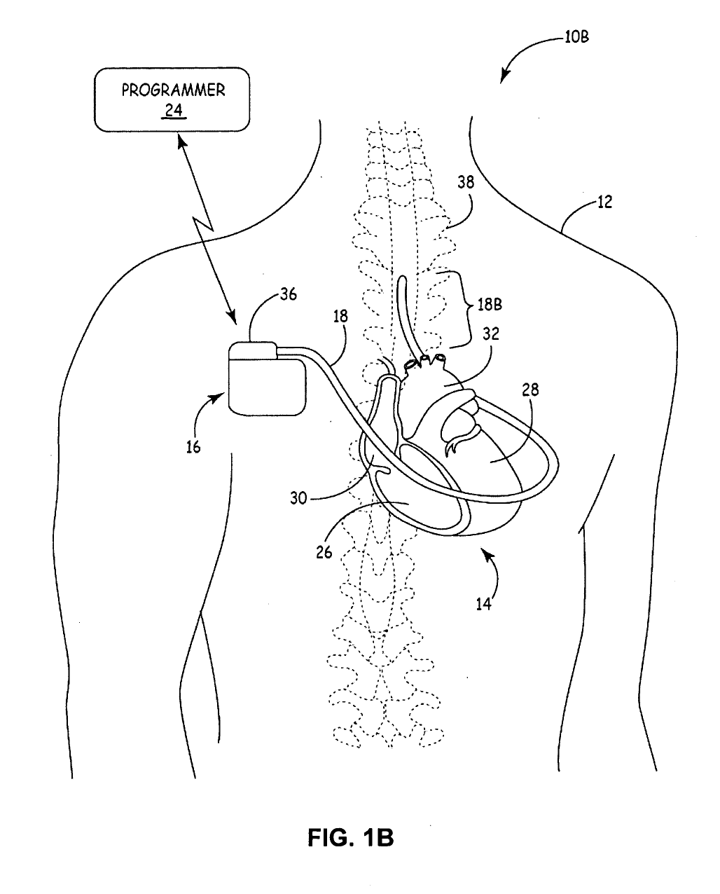

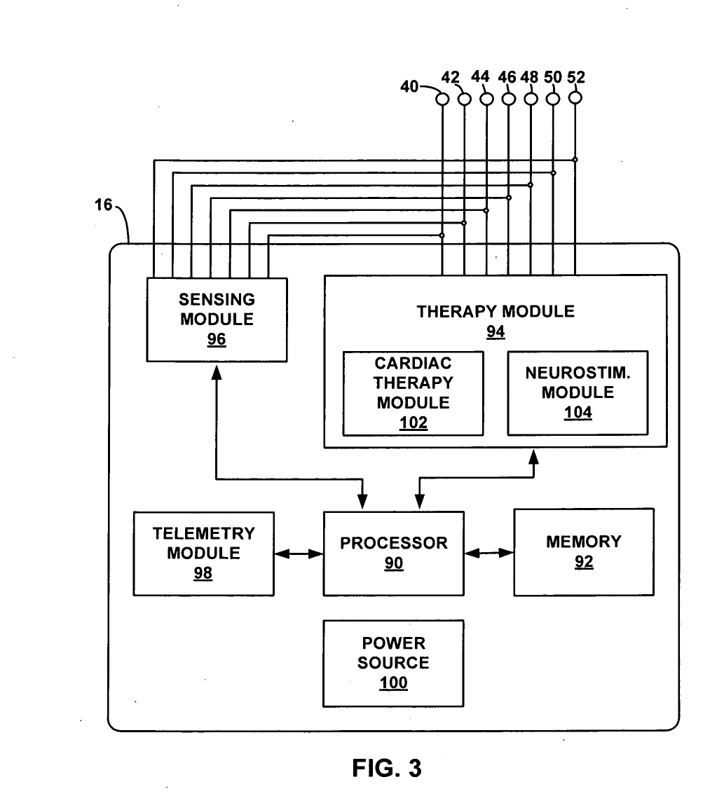

FIG. 1A is a conceptual diagram illustrating an example therapy system 10A that provides stimulation therapy to patient 12. Patient 12 ordinarily, but not necessarily, will be a human. Therapy system 10A includes an implantable medical device (IMD) 16, which is coupled to lead 18, and programmer 24. As described in further detail with respect to FIG. 3, IMD 16 may include a cardiac therapy module and a neurostimulation module in a common housing. The cardiac therapy module may provide functionality similar to an implantable pacemaker, cardioverter, and/or defibrillator, and may generate and deliver electrical signals to heart 14 of patient 12 via extravascular electrodes (not shown in FIG. 1A) carried by lead 18.

The neurostimulation module of IMD 16 may generate and deliver electrical stimulation to modulate the autonomic nervous system of patient 12, e.g., induce a neurohormonal response and/or change in sympathetic and/or parasympathetic autonomic activity. Modulating may include both inhibiting and exciting the autonomic nervous system. For example, the neurostimulation module may generate and deliver stimulation to a nerve or other nonmyocardial tissue site of patient 12, e.g., proximate a vagus nerve, a spinal cord or heart 14 of patient 12, via intravascular and/or extravascular electrodes coupled to lead 18. A nonmyocardial tissue site may include a tissue site that does not include cardiac muscle (e.g., the myocardium). For example, a nonmyocardial tissue site may be proximate a muscle other than cardiac muscle, an organ other than heart 14, or neural tissue. The nonmyocardial tissue site may include extravascular tissue sites or intravascular tissue sites.

In some examples, delivery of neurostimulation to a tissue site, such as a nonmyocardial tissue site or a nonvascular cardiac tissue site, may help modulate an autonomic nervous system of patient 12. In some examples, the neurostimulation module of IMD 16 may deliver electrical stimulation therapy to a nerve of patient 12 via a lead implanted within vasculature (e.g., a blood vessel) of patient 12. In some examples, the neurostimulation module may generate electrical stimulation that is delivered to peripheral nerves that innervate heart 14, or fat pads on heart 14 that may contain nerve bundles. In the example shown in FIG. 1A, electrodes of lead 18 are positioned outside the vasculature of patient 12 and positioned to deliver electrical stimulation to target tissue site 20 proximate a vagus nerve of patient 12. Stimulation may be delivered to extravascular tissue sites, for example, when lead 18 is not implanted within vasculature, such as within a vein, artery or heart 14. In other examples, stimulation may be delivered to a nonmyocardial tissue site via electrodes of an intravascular lead that is implanted within vasculature.

In the example shown in FIG. 1A, the cardiac therapy module and the neurostimulation module are electrically coupled to a common medical lead 18 either directly or indirectly (e.g., via a lead extension). For example, as illustrated in FIG. 1A, the cardiac therapy module may deliver stimulation via one or more electrodes on proximal portion 18A of lead 18 positioned proximate to heart 14, and the neurostimulation module may deliver stimulation via one or more electrodes on a distal portion 18B of lead 18 positioned proximate to target stimulation site 20 near the vagus nerve of patient 12. As described in further detail below, in some examples, the cardiac therapy module and the neurostimulation module may be electrically coupled to different electrodes carried by lead 18.

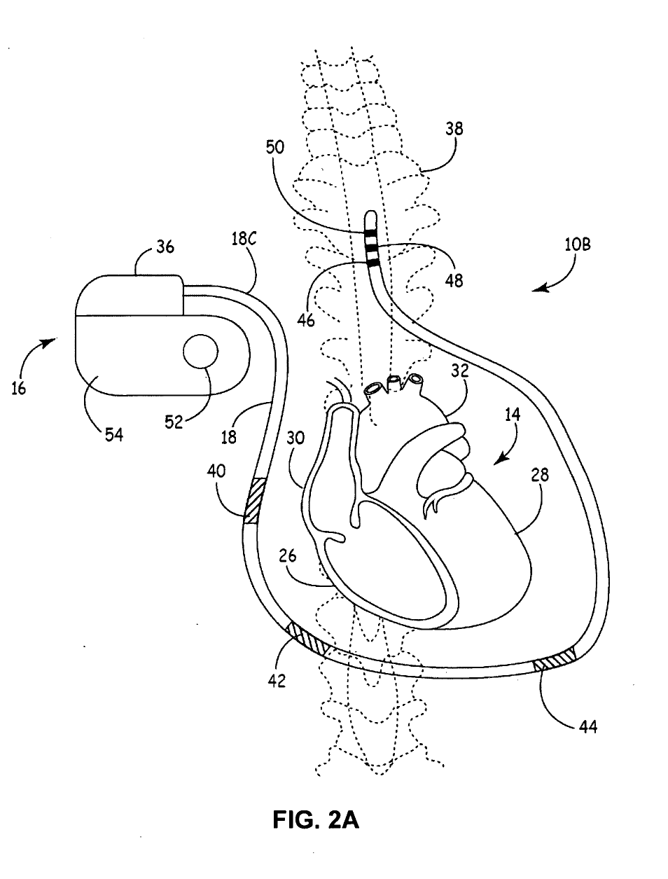

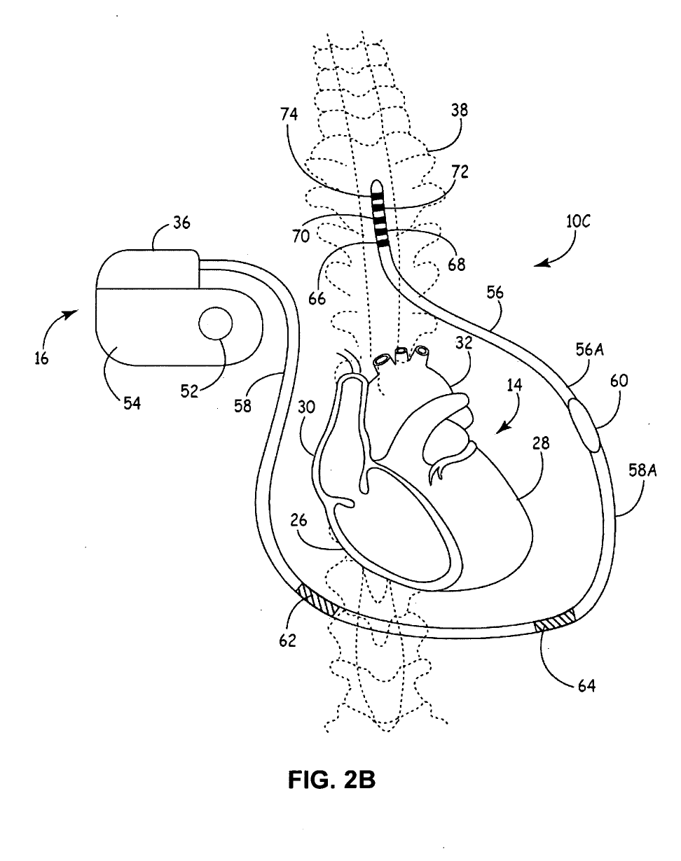

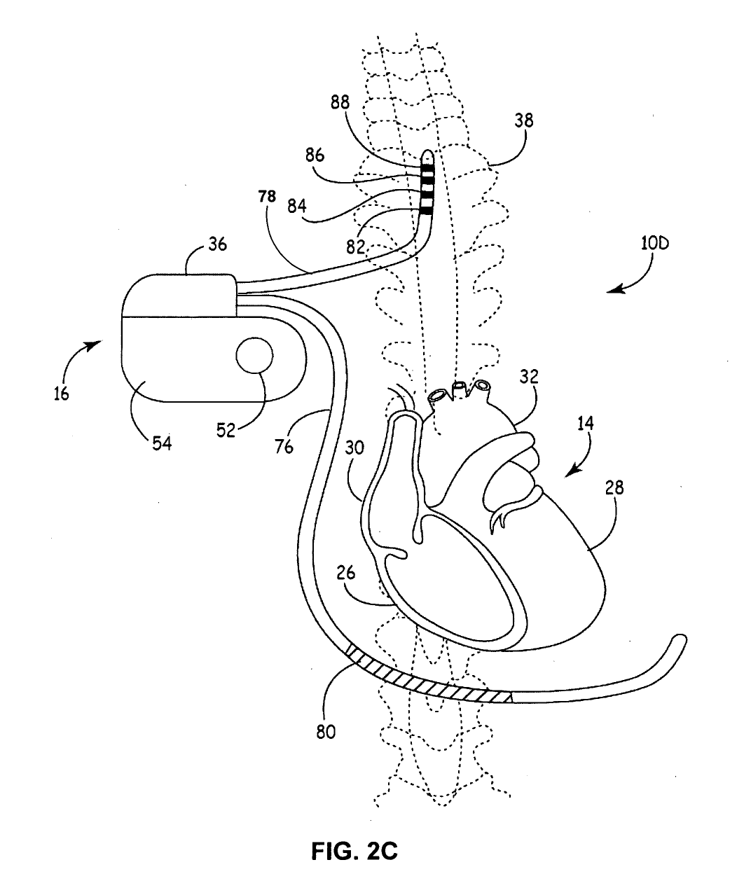

In other example therapy systems, IMD 16 may be coupled to two or more leads, either directly or indirectly (e.g., via a lead extension and/or the leads may be coupled to IMD 16 in series). For example, the neurostimulation module of IMD 16 may be coupled to two leads in order to provide bilateral or multi-lateral stimulation. However, in some examples, due to the placement of electrodes of lead 18 within patient 12, the neurostimulation module of IMD 16 may provide bilateral or multi-lateral stimulation via electrodes of a single lead 18. As described with respect to FIGS. 2B and 2C, in some examples, the cardiac therapy and neurostimulation modules of IMD 16 may deliver therapy to patient 12 with separate leads. That is, in some examples, the cardiac therapy module may deliver electrical stimulation therapy via extravascular electrodes coupled to one lead, and the neurostimulation therapy may deliver therapy via intravascular and/or extravascular electrodes coupled to a second lead that is separate from the first.

IMD 16 may sense electrical cardiac signals attendant to the depolarization and repolarization of heart 14 via extravascular electrodes carried by lead 18. These signals may be referred to as electrocardiogram (ECG) signals or electrogram (EGM) signals. In some examples, the cardiac therapy module of IMD 16 may provide pacing pulses to heart 14 based on the electrical cardiac signals. Pacing therapies may include anti-tachyarrhythmia pacing and/or pacing therapies designed to prevent ventricular tachycardia, ventricular fibrillation, atrial tachycardia, and/or atrial fibrillation. The configurations of electrodes used by IMD 16 for sensing and pacing may be unipolar or bipolar. The cardiac therapy module of IMD 16 may also provide defibrillation therapy and/or cardioversion therapy via electrodes located on lead 18 and/or one or more electrodes on an outer housing of IMD 16. IMD 16 may detect an arrhythmia of heart 14, such as fibrillation of ventricles 26 and 28, and the cardiac therapy module of IMD 16 may deliver defibrillation therapy to heart 14 in the form of electrical pulses based on the detection of the arrhythmia. In some examples, the cardiac therapy module of IMD 16 may be programmed to deliver a progression of therapies, e.g., pulses with increasing energy levels, until a fibrillation of heart 14 is stopped. IMD 16 may detect fibrillation employing one or more fibrillation detection techniques known in the art.

The neurostimulation module of IMD 16 may provide a programmable stimulation signal, e.g., in the form of electrical pulses or a continuous signal, that is delivered to target stimulation site 20 by lead 18, and more particularly, via one or more stimulation electrodes carried by lead 18 and/or the one or more electrodes on the outer housing of IMD 16. In some examples, lead 18 may also carry sense electrodes to permit IMD 16 to sense electrical signals from target stimulation site 20. Like the cardiac therapy module, the neurostimulation module may deliver stimulation based on the electrical cardiac signals. As one example, the neurostimulation module may deliver neurostimulation signals at specific timing related to cues of the EGM (or ECG) signal profile, such as during an arrhythmia. The neurostimulation signal may be modulated, such as rate or duration of pulses, in accordance with presence and/or type of arrhythmia.

In some examples, the neurostimulation module may deliver therapy in advance of any apparent need of arrhythmia correction, such as prior to confirmation of the existence of an arrhythmia. When an arrhythmia is detected, the neurostimulation module may deliver neurostimulation therapy to attempt to correct the arrhythmia, and the cardiac module may deliver a defibrillation shock if the neurostimulation therapy is unsuccessful in correcting the arrhythmia. Attempting to correct the arrhythmia with neurostimulation prior to delivering a defibrillation shock may help avoid delivering unnecessary defibrillation shocks to patient 12.

In some examples, the neurostimulation module may deliver neurostimulation to reduce the severity of a sensed cardiac event, e.g., arrhythmia, fibrillation, electromechanical disassociation. The neurostimulation module may deliver therapy prior to, during, and/or after therapy delivery by the cardiac module of IMD 16, e.g., delivery of a defibrillation shock. The neurostimulation module of IMD 16 may also deliver neurostimulation signals to reduce the risks associated with the cardiac therapy delivered by the cardiac module of IMD 16. As one example, the neurostimulation module may reduce the risks associated with antitachycardia pacing delivered by the cardiac module by improving the heart's rhythm and reducing the chance of antitachycardia pacing inadvertently inducing a heart rhythm problem. In various examples, IMD 16 may deliver pacing that includes one or both of anti-tachycardia pacing (ATP) and cardiac resynchronization therapy (CRT).

In the example shown in FIG. 1A, the neurostimulation module of IMD 16 provides electrical stimulation therapy of a parasympathetic nerve of the autonomic nervous system, such as a vagus nerve, of patient 12. Stimulation of a parasympathetic nerve of patient 12 may help slow intrinsic rhythms of heart 14, which may both facilitate antitachyarrhythmia therapy, e.g., antitachycardia pacing, cardioversion or defibrillation, delivered by the cardiac therapy module of IMD 16. In other examples, the neurostimulation module of IMD 16 may deliver stimulation to increase the heart rate of heart 14, e.g., by delivering stimulation signals to other locations of patient 12 and/or changing one or more parameters of the neurostimulation signal. In this way, neurostimulation by the neurostimulation module of IMD 16 may help control a heart rate of patient 12 and may provide therapy that complements the cardiac rhythm therapy delivered by the cardiac therapy module. In other examples, neurostimulation by the neurostimulation module of IMD 16 may not have an effect on the heart rate of patient 12. For example, neurostimulation by the neurostimulation module of IMD 16 may affect vascular tone, which may improve a condition of patient 12, such as heart failure status, and/or complement the cardiac rhythm therapy delivered by the cardiac therapy module. For example, the neurostimulation module of IMD 16 may deliver stimulation signals to regulate blood pressure, e.g., increase or decrease blood pressure.

In some examples, cardiac therapy by the cardiac module of IMD 16 may complement the neurostimulation by the neurostimulation module of IMD 16. For example, the neurostimulation module may deliver therapy, and a patient response to the neurostimulation therapy may be monitored to determine if the cardiac therapy should deliver cardiac therapy. The patient response may include, for example, a cardiac response (e.g., that indicates cardiac capture), a neural response (e.g., a neural signal that indicates nerve capture), or any other suitable physiological response. The neurostimulation module may deliver neurostimulation therapy to, for example, modify a heart rate, improve a heart failure status, prevent an arrhythmia, terminate an arrhythmia, or modify a blood pressure of the patient. IMD 16 may sense the patient's response to the neurostimulation therapy delivered by the neurostimulation module, e.g., via signals indicative of cardiac electrical activity, blood pressure, or tissue perfusion, and the cardiac module of IMD 16 may apply cardiac therapy based on the sensed response to the neurostimulation therapy.

In other examples, electrodes of lead 18 may be positioned to deliver electrical stimulation to any other suitable nerve, organ, muscle or muscle group in patient 12, which may be selected based on, for example, a therapy program selected for a particular patient. In some examples, the neurostimulation module of IMD 16 may deliver electrical stimulation to other sympathetic or parasympathetic nerves, peripheral nerves, cardiac fat pads, renal nerves, baroreceptors, the carotid sinus or a cardiac branch of the vagal trunk of patient 12 in order to facilitate the delivery of therapy by the cardiac therapy module of IMD 16.

Like the cardiac therapy module, the neurostimulation module of IMD 16 may also adjust and/or deliver therapy in response to a sensed cardiac event, e.g., depolarization or repolarization of heart 14, or an arrhythmia. For example, if the neurostimulation module delivers therapy to control heart rate, the sensed depolarization and repolarization of heart 14 may provide feedback for therapy adjustment. As another example, the neurostimulation module may deliver therapy to control heart rate in response to a sensed arrhythmia, which may include abnormal heart rhythms such as bradycardia, tachycardia or fibrillation. A sensing module of IMD 16 may detect an arrhythmia using any suitable technique, such as techniques that rely on detected electrical cardiac signals generated by the depolarization and repolarization of heart 14, heart rate, or hemodynamic parameters, such as blood oxygen levels, blood pressure, stroke volume, and the like. IMD 16 may also sense other parameters, such as tissue perfusion, transthoracic impedance, cardiac impedance, and/or acoustic cardiac sounds, and the cardiac and/or neurostimulation modules of IMD 16 may, additionally or alternatively, adjust and/or deliver therapy in response to these sensed parameters.

In some cases, the delivery of neurostimulation by the neurostimulation module of IMD 16 may help eliminate or reduce the demands of pacing or defibrillation provided by the cardiac therapy module. For example, in some examples, neurostimulation may decrease the threshold levels, e.g., voltage or current threshold levels, required to defibrillate heart 14 of patient 12 by, for example, reducing the threshold levels that are needed for the cardiac therapy module to terminate the fibrillation of heart 14. This may help the cardiac therapy module terminate fibrillation at a threshold level less than the maximum output of the cardiac module, IMD 16 of extravascular therapy system 10A may need to deliver higher amplitude therapy to defibrillate heart 14 with extravascular electrodes that are positioned outside of heart 14 compared to therapy systems that utilize intravascular electrodes. Thus, reducing the defibrillation threshold levels may be particularly useful in the therapy system 10A, which utilizes extravascular electrodes to deliver defibrillation shocks. In addition to helping ensure the threshold level is within the stimulation output range of IMD 16, reducing a defibrillation threshold level may reduce the patient that patient 12 experiences subsequent to receiving a defibrillation shock.

In other examples, the neurostimulation module of IMD 16 may deliver therapy to reduce the recovery time after defibrillation, for example, by improving cardiac output and/or stroke volume. For example, the neurostimulation module of IMD 16 may deliver stimulation signals subsequent to the delivery of a defibrillator shock delivered by the cardiac module of IMD 16. The neurostimulation therapy may aid in returning heart 14 to a normal rhythm. Thus, the neurostimulation therapy may also reduce the need for post-shock pacing of heart 14 by the cardiac module of IMD 16. Reducing the need for post-shock pacing of heart 14 may be particularly important in examples in which the cardiac module of IMD 16 does not deliver pacing therapy to heart 14, e.g., cardiac modules that solely deliver cardioversion and/or defibrillation therapy.

In other examples, the delivery of neurostimulation by the neurostimulation module of IMD 16 may help prevent or reduce the tendency of heart 14 to beat irregularly, which may reduce the amount of energy consumed by the cardiac therapy module of IMD 16 to sustain a regular heart beat. Thus, in some examples, the neurostimulation module of IMD 16 may deliver therapy when the cardiac therapy module is not delivering therapy. This may help reduce the frequency with which the cardiac therapy module generates and delivers therapy to terminate an arrhythmia of heart.

In some examples, depending upon the neurostimulation target, the delivery of electrical stimulation by the neurostimulation module may also mitigate perceptible discomfort generated from the delivery of pacing pulses or cardioversion/defibrillation shocks by the cardiac therapy module. For example, if the neurostimulation module delivers electrical stimulation to a spinal cord of patient 12, as shown and described with respect to FIGS. 2B and 2C, the neurostimulation may produce paresthesia, which may help reduce the discomfort felt by patient 12 from the delivery of stimulation by the cardiac therapy module.

In general, the cardiac and neurostimulation modules of IMD 16 may deliver therapy at substantially the same time and/or at different times. For example, a processor within IMD 16 may control the cardiac and neurostimulation modules to deliver therapy substantially concurrently in response to detection of fibrillation. Additionally or alternatively, the neurostimulation module may provide neurostimulation prior to and/or subsequent to a defibrillation shock delivered by the cardiac therapy module. Other functions of therapy system 10A, such as sensing electrical signals, may be performed coincident and/or in alternation with therapy delivery by the cardiac therapy module and/or neurostimulation module of IMD 16.

In the example of FIG. 1A, IMD 16 has been implanted in the chest cavity, e.g., in the intraclavicular, subclavicular, or mammary area, of patient 12. Other implant locations are also contemplated, such as in the back or abdomen of patient 12. IMD 16 may be subcutaneously or submuscularly implanted in the body of a patient 12 at any appropriate location. Upon implantation of IMD 16, proximal end 18C of lead 18 may be both electrically and mechanically coupled to connector 36 of IMD 16 either directly or indirectly (e.g., via a lead extension). In particular, conductors disposed in the lead body may electrically connect stimulation electrodes (and sense electrodes, if present) of lead 18 to IMD 16.

In some examples, lead 18 may be subcutaneously implanted in patient 12. For example, lead may be tunneled between IMD 16, heart 14, and target stimulation site 20 near the vagus nerve of patient 12. By tunneling lead 18 proximate to but outside of heart 14, the cardiac therapy module of IMD 16 may deliver therapy to heart 14 without requiring lead 18 to be implanted in heart 14. Proximal portion 18A of lead 18 may include one or more extravascular electrodes that the cardiac therapy module may utilize to deliver therapy to heart 14. For example, proximal portion 18A of lead 18 may include extravascular electrodes that are positioned proximate to right atrium 30, right ventricle 26, left atrium 32, and/or left ventricle 28. Distal portion 18B may include one or more electrodes with which the neurostimulation module of IMD 14 may deliver stimulation to target stimulation site 20 near the vagus nerve of patient 12. Various electrode configurations of lead 18 are described in further detail with respect to FIGS. 2A-2C.

In other examples, as shown in FIG. 1B, lead 18 may be positioned to allow the neurostimulation module of IMD 12 to deliver electrical stimulation to spinal cord 38 of patient 12. In some examples, lead 18 may be positioned within patient 12 such that at least some electrodes on distal portion 18B of lead 18 are positioned in the intrathecal space or epidural space of spinal cord 38 near the spinal segments T1-T6, or, in some examples, adjacent nerves that branch off of spinal cord 38. In one example technique for implanting lead 18 in patient 12, lead 18 may be tunneled from IMD 16 to position electrodes proximate heart 14, and then further tunneled in a ventral direction to the back of patient 12 in order to access spinal cord 38. In some examples, electrodes of lead 18 may be positioned proximate to spinal cord 38 at approximately the same anteroposterior position as heart 14.

Lead 18 may be introduced into spinal cord 38 in the thoracic region, as shown in FIG. 1B. In other examples, lead 18 may be introduced into spinal cord 38 in the cervical or lumbar regions. Stimulation of spinal cord 38 or nerves branching therefrom by the neurostimulation module of IMD 16 may help prevent or mitigate occurrences of tachyarrhythmias and may reduce the level of aggressiveness of the cardiac therapy, such as pacing, cardioversion or defibrillation, delivered by the cardiac therapy module of IMD 16. In this manner, the cardiac and neurostimulation modules of IMD 16 may operate in conjunction with each other to help prevent arrhythmias of heart 14 of patient 12, as well as to terminate detected arrhythmias.

In some examples, programmer 24 may be a handheld computing device or a computer workstation. Programmer 24 may include a user interface that receives inputs from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display.

A user, such as a physician, technician, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of IMD 16.

For example, the user may use programmer 24 to retrieve information from IMD 16 regarding the rhythm of heart 14, trends therein over time or tachyarrhythmia episodes. As another example, the user may use programmer 24 to retrieve information from IMD 16 regarding other sensed physiological parameters of patient 12, such as electrical depolarization/repolarization signals from the heart (e.g., EGM signals), intracardiac or intravascular pressure, activity, posture, respiration or thoracic impedance.

The user may use programmer 24 to program a therapy progression for IMD 16. As one example, programmer 24 may select electrodes (i.e., an electrode combination) with which the cardiac therapy module of IMD 16 may deliver defibrillation pulses, select waveforms for the defibrillation pulse or select or configure a fibrillation detection algorithm for IMD 16. The user may also use programmer 24 to program aspects of other therapies provided by the cardiac therapy module of IMD 16, such as cardioversion or pacing therapies.

The user may also use programmer 24 to program aspects of the neurostimulation module. The therapy parameters for the neurostimulation module of IMD 16 may include an electrode combination for delivering neurostimulation signals, as well as an amplitude, which may be a current or voltage amplitude, and, if the neurostimulation module delivers electrical pulses, a pulse width, and a pulse rate for stimulation signals to be delivered to patient 12. The electrode combination may include a selected subset of one or more electrodes located on implantable lead 18 coupled to IMD 16 and/or a housing of IMD 16. The electrode combination may also refer to the polarities of the electrodes in the selected subset. By selecting particular electrode combinations, a clinician may target particular anatomic structures within patient 12. In addition, by selecting values for amplitude, pulse width, and pulse rate, the physician can attempt to generate an efficacious therapy for patient 12 that is delivered via the selected electrode subset.

As another example, the user may use programmer 24 to retrieve information from IMD 16 regarding the performance or integrity of IMD 16 or other components of the relevant therapy system 10A or 10B, such as lead 18 or a power source of IMD 16. With the aid of programmer 24 or another computing device, a user may select values for therapy parameters for controlling therapy delivery by the cardiac and neurostimulation modules of IMD 16. The values for the therapy parameters may be organized into a group of parameter values referred to as a “therapy program” or “therapy parameter set.” “Therapy program” and “therapy parameter set” are used interchangeably herein.

Programmer 24 may communicate with IMD 16 via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

FIG. 2A is a conceptual diagram illustrating IMD 16 and lead 18 of therapy system 10B in greater detail. Lead 18 may be electrically coupled to the cardiac therapy module, the neurostimulation module, a sensing module, or other modules IMD 16 via connector block 36. For example, the proximal end of lead 18 may include electrical contacts that electrically couple to electrical contacts within connector block 36, which provides electrical connections to the modules within IMD 16. In addition, in some examples, lead 18 may be mechanically coupled to connector block 36 with the aid of set screws, connection pins or another suitable mechanical coupling mechanism. In some examples, lead 18 may include an elongated insulative lead body, which may carry a number of conductors that are electrically coupled to a respective one of the electrodes carried by lead 18. The conductors may be disposed in a common lead body in order to help define a lead 18 that is relatively easy to manipulate and implant within patient 12. The conductors of lead 18 may be electrically coupled to circuitry within IMD 16 via the electrical connections provided by connector block 36. In some examples, the conductors may be concentrically coiled and separated from one another by tubular insulative sheaths. Other lead configurations are also contemplated.

In the illustrated example, lead 18 includes electrodes 40, 42, 44, 46, 48, and 50. Each of the electrodes 40, 42, 44, 46, 48, and 50 may be electrically coupled to a respective one of the conductors within the lead body of lead 18, and thereby coupled to respective ones of the electrical contacts on proximal end 18C of lead 18. Electrodes 40, 42, 44, 46, 48, and 50 may be fabricated from any suitable electrically conductive material, such as, but not limited to, platinum, platinum alloy or other materials known to be usable in implantable stimulation electrodes. In the example illustrated in FIG. 2A, lead 18 is positioned within patient 12 such that electrodes 40, 42, and 44 are proximate to heart 14. In this way, the cardiac therapy module of IMD 16 may deliver electrical stimulation therapy to heart 14 via at least electrodes 40, 42, 44 of lead 18. Lead 18 is also implanted within patient 12 such that electrodes 46, 48, and 50 are positioned proximate to spinal cord 38. The neurostimulation module within IMD 16 may deliver electrical stimulation to spinal cord 38 via at least electrodes 46, 48, 50.

Electrodes 40, 42, and 44 are implanted proximate to, but outside of heart 14. Therefore, electrodes 40, 42, and 44 may be referred to as extravascular electrodes. An extravascular electrode may comprise an electrode that is not implanted within heart 14 or within an artery or other vasculature of the patient 12. For example, electrodes 40, 42, and 44 may comprise subcutaneous, submuscular, epicardial, and/or intramural electrodes. In some examples, electrodes 46, 48, and 50 may also be extravascular electrodes.

In some examples, as illustrated in FIG. 2A, IMD 16 may include one or more housing electrodes, such as housing electrode 52, which may be formed integrally with an outer surface of hermetically-sealed housing 54 of IMD 16 or otherwise coupled to housing 54. In some examples, housing electrode 52 is defined by an uninsulated portion of an outward facing portion of housing 54 of IMD 16. In some examples, housing electrode 52 comprises substantially all of housing 54. Other divisions between insulated and uninsulated portions of housing 54 may be employed to define two or more housing electrodes. For example, housing 54 may include three housing electrodes 52. In examples in which IMD 16 includes two or more housing electrodes, the cardiac and/or neurostimulation module may deliver a stimulation signal between two housing electrodes.

One or more of housing electrodes, such as housing electrode 52, may be embedded in a coating of housing 54. IMD 16 may also include one or more electrodes separate from, but attached to housing 54. For example, IMD 16 may include a modified housing electrode that is suspended from housing 52 via a connector element, such as an insulated electrical conductor. IMD 16 may also include one or more electrodes on an outer surface of connector block 36.

A sensing module of IMD 16 may sense electrical physiological signals of patient 12 via two or more electrodes 40, 42, 44, 46, 48, 50, 52. The electrical signals may be conducted to IMD 16 via lead 18, or, in the case of housing electrode 52, via a conductor within housing 54 and used by the cardiac and neurostimulation modules of IMD 16 to modulate therapy. As one example, electrodes 40, 42, and 44 may sense signals attendant to the depolarization and repolarization of heart 14.

The cardiac therapy module of IMD 16 may deliver pacing pulses via any combination of electrodes 40, 42, and 44 and housing electrode 52, e.g., any unipolar or bipolar electrode configuration, to cause depolarization of cardiac tissue of heart 14. The cardiac therapy module of IMD 16 may alternatively or additionally deliver defibrillation and/or cardioversion pulses to heart 14 via electrodes 40, 42, 44, and 52. Electrodes 40, 42, and 44 may comprise elongated electrodes that take the form of coil electrodes. Such coil electrodes may be useful in delivering high energy defibrillation pulses to heart 14.

The neurostimulation module of IMD 16 may deliver neurostimulation via any combination of electrodes 46, 48, and 50 and housing electrode 52. In the example shown in FIG. 2A, the neurostimulation module of IMD 16 delivers neurostimulation to spinal cord 38 of patient 12. This may be referred to as spinal cord stimulation (SCS). In some examples, the neurostimulation module may deliver a stimulation signal between one of electrodes 46, 48, or 50 and housing electrode 52, i.e., in a unipolar configuration. As another example, the neurostimulation module may deliver a stimulation signal between a plurality of electrodes 46, 48, and 50, e.g., in a bipolar configuration. In some examples, electrodes 46, 48, and 50 may take the form of ring, partial ring or segmented electrodes. Ring electrodes may extend substantially completely around the lead body of lead 18, whereas partial ring and segmented electrodes may extend partially around the lead body of lead 18. A plurality of segmented electrodes may be located at substantially the same axial position of lead 18, e.g., to form a row of segmented electrodes. Ring electrodes may be relatively simple to program and are capable of delivering an electrical field to any tissue adjacent to the respective electrode 46, 48, and 50. In other examples, at least one of the electrodes 46, 48, and 50 may have a different configuration. For examples, in some examples, at least one of the electrodes 46, 48, and 50 may have a complex electrode array geometry that is capable of producing shaped electrical fields. The complex electrode array geometry may include multiple electrodes (e.g., partial ring or segmented electrodes) around the outer perimeter of each lead 20, rather than one ring electrode.

In some examples, a first subset of electrodes 40, 42, 44, 46, 48, 50, 52 that are used to deliver cardiac rhythm therapy to patient 12 and a second subset of 40, 42, 44, 46, 48, 50, 52 that are used to deliver neurostimulation to patient 12 may share a common return electrode. For example, housing electrode 52 may serve as a return electrode for both cardiac rhythm and neurostimulation therapies. As another example, cardiac rhythm and neurostimulation therapies may share one or more other electrodes, such as electrodes 40, 42, 44, 46, 48, and/or 50. Delivering cardiac rhythm and neurostimulation therapies with one or more of the same electrodes, e.g., one or more of electrodes 40, 42, 44, 46, 48, 50 and 52, may be particularly useful in examples in which IMD 16 delivers neurostimulation subcutaneously, transcutaneously, and/or in the form of peripheral nerve field stimulation (PNFS). In some examples, IMD 16 delivers different waveforms, e.g., sinusoidal, square, or pulse, to one or more of the same electrodes to elicit different responses from patient 12 (FIG. 1) for the neurostimulation and the cardiac rhythm therapy. More specifically, IMD 16 may deliver cardiac pacing therapy using a first waveform and neuromodulation therapy using a second, different waveform.

In some examples, electrodes 46, 48, and 50 may be electrically isolated from electrodes 40, 42, and 44. For example, lead 18 and/or IMD 16 may be configured to isolate any high energy shock that the cardiac therapy module delivers to electrodes 40, 42, 44, and 52 from electrodes 46, 48, and 50. IMD 16 may include circuitry configured to achieve such isolation.

In some examples, the neurostimulation module of IMD 16 may utilize electrodes 40, 42, and 44 to deliver neurostimulation. For example, the neurostimulation module of IMD 16 may deliver stimulation via any combination of electrodes 40, 42, 44, 46, 48, and 50 and housing electrode 52. The neurostimulation module of IMD 16 may utilize electrodes 40, 42, and 44, for example, to stimulate the cardiac fat pads on heart 14.

The number, configuration, and type of electrodes 40, 42, 44, 46, 48, 50 and 52 shown in FIG. 2A are merely exemplary. In other examples, lead 18 may include any number, configuration, and type of electrodes 40, 42, 44, 46, 48, 50, and 52. For example, lead 18 may include one or more additional electrodes proximate to heart 14, e.g., proximate to left atrium 32 As another example, lead 18 may include a fewer or a greater number of electrodes proximate to spinal cord 38. A greater number of electrodes may permit the neurostimulation module of IMD 16 to stimulate fewer or additional locations of spinal cord 38. Also, electrodes 40, 42, 44, 46, 48, 50, 52 may be positioned at different locations on lead 18 or housing 54, e.g., to permit stimulation at different locations of patient 12. In the example illustrated in FIG. 2A, therapy system 10B includes a single lead 18. In other examples, therapy system 10B may also include two or more leads.

FIG. 2B is a conceptual diagram illustrating another example of therapy system 10C, which is similar to therapy systems 10A and 10B of FIGS. 1A, 1B, and 2A, but includes a neurostimulation extension lead 56 coupled to a cardiac lead 58 via a lead connector 60. In the example illustrated in FIG. 2B, cardiac lead 58 includes extravascular electrodes 62 and 64 positioned proximate to heart 14 and, more specifically, proximate to right ventricle 26 and left ventricle 28 of heart 14, respectively. The cardiac therapy module of IMD 16 may deliver pacing, cardioversion, and/or defibrillation therapy, e.g., in the form electrical stimulation signals, to heart 14 using any combination of electrodes 62, 64, and 52.

A distal end 58A of cardiac lead 58 may be mechanically and electrically coupled to a proximal end 56A of neurostimulation lead 56 via lead connector 60. Lead connector 60 may include electrical contacts similar to those of connector block 36. For example, electrical contacts at distal end 58A of cardiac lead 58 may electrically couple to electrical contacts within lead connector 60. Electrical contacts at a proximal end 56A of neurostimulation lead 56 may also couple to electrical contacts within lead connector 60 to electrically couple neurostimulation lead 56 to cardiac lead 58 and, ultimately, IMD 16 via conductors carried within cardiac lead 58.

The electrical contacts at distal end 58A of cardiac lead 58 may be electrically isolated from other conductors within cardiac lead 58 that electrically couple electrodes 62 and 64 to IMD 16. For example, the conductors within cardiac lead 58 that electrically couple electrodes 62 and 64 to IMD 16 may be sufficiently insulated from the conductors that couple to neurostimulation lead 56 at lead connector 60. In some examples, IMD 16 may include isolation circuitry to further aid in electrically isolating cardiac electrodes 62 and 64 from neurostimulation electrodes 66, 68, 70, 72, and 74 of neurostimulation lead 56, as described above. As one example, the isolation circuitry may create an electrical disconnect between neurostimulation lead 56 and IMD 16 when the cardiac therapy module delivers a high energy defibrillation pulse via a combination of electrodes 62, 64, and/or 52.

Electrodes 66, 68, 70, 72, and 74 may be positioned proximate to spinal cord 38 to deliver spinal cord stimulation. As described with respect to FIG. 2A, the neurostimulation module of IMD 16 may deliver a bipolar stimulation signal between two electrodes proximate to spinal cord 38, e.g., between two of electrodes 66, 68, 70, 72, and 74. Alternatively, the neurostimulation module of IMD 16 may deliver unipolar stimulation between housing electrode 52 and an electrode proximate to spinal cord 38, e.g., electrode 66, 68, 70, 72 or 74. In some examples, the neurostimulation module of IMD 16 may utilize one or more of electrode 62 and 64 to deliver neurostimulation, e.g., to the cardiac fat pads, in a unipolar or multipolar configuration. The configuration of electrodes 62, 64, 66, 68, 70, 72, and 74 illustrated in FIG. 2B is for purposes of example. In other examples, cardiac lead 58 and/or neurostimulation lead 56 may include any number, type or configuration of electrodes.

Therapy systems 10C, as well as the other therapy systems described herein, may take advantage of the placement of cardiac lead 58 within patient 12 to provide the additional cardiac benefits that may result from the delivery of neurostimulation. In particular, systems that only include a cardiac therapy module may already include cardiac lead 58 that carries extravascular electrodes 62, 64 to provide pacing, cardioversion, and/or defibrillation pulses to heart 14. Thus, therapy system 10C leverages the existing location of a medical device and cardiac lead 58 to provide IMD 16 including both a cardiac therapy module and a neurostimulation module, as well as neurostimulation lead 56 as an extension on cardiac lead 58. In this way, therapy system 10C may provide further benefits to patient 12 in a minimally invasive manner compared to existing cardiac therapy systems that include extravascular cardiac therapy electrodes.

A neurostimulation lead 56 that is mechanically coupled to cardiac lead 58 via lead extension 60 may support a relatively flexible implantation process. For example, a clinician may implant leads 56 and 58 in any order. This flexibility may allow easier and/or more precise electrode placement. Moreover, the modular design of neurostimulation lead 56 also provides the flexibility of adding neurostimulation lead 56 to a therapy system at a later time. For example, IMD 16 and cardiac lead 62 may be implanted in a first implantation procedure and the therapy effectiveness may be evaluated. If needed or desired, neurostimulation lead 56 may be added in a second implantation procedure at a later time. Distal end 58A of cardiac lead 58 and/or connector 60 may be sealed, e.g., by a cap, biocompatible material, or other mechanism, to avoid fluid ingress when neurostimulation lead 56 is not used, e.g., not connected to cardiac lead 58 via connector 60.

FIG. 2C is a conceptual diagram illustrating another example of therapy system 10D, which is similar to therapy systems 10A-10C of FIGS. 1A, 1B, 2A, and 2B, but includes two separate, physically disconnected leads 76 and 78 that couple to IMD 16 in parallel. In some examples, leads 76, 78 may be attached at one or more points along the lengths of the leads 76, 78, e.g., via a sheath, adhesive, and/or other attachment element. This may be useful for fixing the position of leads 76, 78 relative to each other. Lead 76 includes electrode 80 positioned proximate to heart 14. The cardiac therapy module of IMD 16 may, for example, deliver a defibrillation pulse to heart 14 via electrodes 80 and 52. Lead 78 includes electrodes 82, 84, 86, and 88 positioned proximate to spinal cord 38. The neurostimulation module of IMD 16 may, for example, deliver SCS via any combination of electrodes 82, 84, 86, 88, and 52.

In the example illustrated in FIG. 2C, leads 76 and 78 may be utilized to deliver different therapies. For example, the cardiac therapy module of IMD 16 may deliver therapy directly to heart 14, e.g., pacing, cardioversion, and/or defibrillation therapy, with electrode 80 of lead 76, and the neurostimulation module may deliver stimulation signals to spinal cord 38 with any one or more of the electrodes 80, 82, 84, 86, and 88 of lead 78 to deliver stimulation signals to spinal cord 38. In other examples, one bifurcated lead may be provided instead of individual leads 76 and 78. As another example, multiple leads may be utilized by the neurostimulation module of IMD 16, e.g., to deliver bilateral stimulation.

FIG. 3 is a functional block diagram of an example configuration of IMD 16, which includes processor 90, memory 92, therapy module 94, sensing module 96, telemetry module 98, and power source 100. The components shown in FIG. 3 may be contained within a common, hermetically sealed housing 54 of IMD 16. Therapy module 94 includes cardiac therapy module 102 and neurostimulation module 104. In the example shown in FIG. 3, therapy module 94 is coupled to electrodes 40, 42, 44, 46, 48, 50 of lead 18 (FIG. 2A) and housing electrode 52. In other examples, however, therapy module 94 may be coupled to electrodes 62, 64 of cardiac lead 58 (FIG. 2B) and electrodes 66, 68, 70, 72, 74 of neurostimulation lead 56 (FIG. 2B), electrode 80 of cardiac lead 76 (FIG. 2C) and electrodes 82, 84, 86, 88 of neurostimulation lead 78 (FIG. 2C), and/or electrodes of other leads.

Memory 92 includes computer-readable instructions that, when executed by processor 90, cause IMD 16 and processor 90 to perform various functions attributed to IMD 16 and processor 90 herein. Memory 92 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processor 90 may include any one or more of a microprocessor, a controller, a digital signal processor (DSP), an application specific integrated circuit (ASIC), a field-programmable gate array (FPGA), or equivalent discrete or integrated logic circuitry. In some examples, processor 90 may include multiple components, such as any combination of one or more microprocessors, one or more controllers, one or more DSPs, one or more ASICs, or one or more FPGAs, as well as other discrete or integrated logic circuitry. The functions attributed to processor 90 herein may be embodied as software, firmware, hardware or any combination thereof. Processor 90 controls therapy module 94 to deliver stimulation therapy according to a selected one or more of therapy programs, which may be stored in memory 92. Specifically, processor 90 may control cardiac therapy module 102 and/or neurostimulation module 104 to deliver electrical signals with the amplitudes, frequency, electrode polarities, and, in the case of stimulation pulses, pulse widths specified by the selected one or more therapy programs.

Therapy module 94 is electrically coupled to electrodes 40, 42, 44, 46, 48, and 50, e.g., via conductors of lead 18, or, in the case of housing electrode 52, via an electrical conductor disposed within housing 54 of IMD 16. Cardiac therapy module 102 and neurostimulation module 104 are disposed within a common housing 54 (FIG. 2A) of IMD 16. Cardiac therapy module 102 and neurostimulation module 104 may each include a respective stimulation generator that generates the electrical stimulation signals for delivery to patient 12. For example, cardiac therapy module 102 and neurostimulation module 104 may be physical separate components of IMD 16 disposed within housing 54. In some examples, e.g., examples in which cardiac therapy module 102 and neurostimulation module 104 deliver stimulation solely in alternation, cardiac therapy module 102 and neurostimulation module 104 may share some or all stimulation generation circuitry. As one example, IMD 16 and/or lead 18 may include switching circuitry, such as one or more multipliers and/or transducers, to allow a single stimulation generator to deliver cardiac and neurostimulation therapy in alternation.

Cardiac therapy module 102 is configured to generate and deliver electrical stimulation therapy to heart 14 (FIG. 1A). For example, a stimulation generator of cardiac therapy module 102 may deliver cardioversion or defibrillation shocks and/or pacing pulses to heart 14 via electrodes 40, 42, and 44 coupled to lead 18 and/or housing electrode 52. In some examples, cardiac therapy module 102 delivers pacing, cardioversion, or defibrillation stimulation in the form of electrical pulses. In other examples, cardiac therapy module 102 may deliver one or more of these types of stimulation in the form of other signals, such as sine waves, square waves, or other substantially continuous time signals.

Neurostimulation module 104 is configured to generate and deliver electrical stimulation therapy to modulate the autonomic nervous system of patient 12. Example stimulation sites for neurostimulation module 104 include, but are not limited to, tissue proximate a vagus nerve or spinal cord 38 of patient 12. For example, a stimulation generator within neurostimulation module 104 may generate stimulation signals that are delivered to spinal cord 38 via electrodes 46, 48, and 50 coupled to lead 18 and/or housing electrode 52. Neurostimulation module 104 may include a single or multi-channel stimulation generator. In particular, the stimulation generator may be capable of delivering, a single stimulation pulse, multiple stimulation pulses, or a continuous signal at a given time via a single electrode combination or multiple stimulation pulses at a given time via multiple electrode combinations. In some examples, however, neurostimulation module 104 may be configured to deliver multiple channels on a time-interleaved basis. In this case, therapy module 94 may include a switching module that serves to time division multiplex the output of the stimulation generator across different electrode combinations at different times to deliver multiple programs or channels of stimulation energy to patient 12.

Processor 90 may control cardiac therapy module 102 and neurostimulation therapy module 104 to coordinate the delivery of electrical stimulation to patient 12. As previously described, in some examples, neurostimulation module 104 may deliver neurostimulation signals to patient 12 at substantially the same time as pacing, cardioversion, and/or defibrillation pulses delivered by cardiac therapy module 102. In those examples, processor 90 may control cardiac therapy module 102 and neurostimulation module 104 to generate and deliver electrical stimulation at substantially the same time. While in some examples, the pulses or other signals delivered by cardiac therapy module 102 and neurostimulation therapy module 104 may not overlap in time, the general time frame that cardiac therapy module 102 and neurostimulation therapy module 104 actively generate and deliver electrical stimulation therapy to patient 12 may overlap.

In other examples, in addition to or instead of delivering neurostimulation signals at substantially the same time that cardiac therapy module 102 delivers stimulation, neurostimulation module 104 may deliver neurostimulation signals to patient 12 prior to or after cardiac therapy module 102 delivers stimulation. In some examples, processor 90 may control neurostimulation module 104 to deliver neurostimulation signals to patient 12 based on physiological parameter values sensed by sensing module 96, which may indicate the presence of an arrhythmia. As one example, processor 90 may control neurostimulation module 104 to deliver neurostimulation signals, e.g., a predetermined number of pulses, when sensing module 96 detects an R-wave of an electrocardiogram (ECG) or electrogram (EGM) signal such that the neurostimulation signals are delivered during the refractory period of the ventricles of heart 14.

In other examples, processor 90 may control neurostimulation module 104 to deliver neurostimulation signals to patient 12 according to a predetermined schedule that is independent of physiological parameter values sensed by sensing module 96. The schedule may be determined by a clinician and stored in memory 92. As previously indicated, the delivery of electrical stimulation by neurostimulation module 104 to a nonmyocardial tissue site to modulate an autonomic system of the patient's nervous system may help regulate the patient's heart rate. Thus, processor 90 may control neurostimulation module 104 to generate and deliver neurostimulation signals to patient 12 as a preventative measure, e.g., to reduce the occurrence of arrhythmias, and, therefore, reduce the frequency with which cardiac therapy module 102 generate and delivers stimulation.

If processor 90 detects an arrhythmia, e.g., based on cardiac signals sensed by sensing module 96, processor 90 may initiate the delivery of stimulation by cardiac therapy module 102. In addition, in some cases, upon the detection of an arrhythmia, processor 90 may control neurostimulation module 104 to generate neurostimulation signals based on a therapy program that is different than the therapy program used by neurostimulation module 104 to generate stimulation signals prior to the detection of the arrhythmia. The therapy programs may differ by at least one stimulation parameter value, such as a voltage or current amplitude, frequency, pulse rate, or pulse width. As another example, the electrode combinations defined by the therapy programs may differ. For example, neurostimulation module 104 may deliver electrical stimulation to patient 12 according to a first subset of electrodes 46, 48, 50, 52 prior to the detection of an arrhythmia, and according to a second subset of electrodes 46, 48, 50, 52 after the detection of an arrhythmia. Utilizing different subsets of electrodes to deliver the neurostimulation may permit neurostimulation module 104 to target different tissue sites, e.g., different parts of a nerve.

In general, if therapy module 94 includes a switch module, processor 90 may control the switch module to select, e.g., via a data/address bus, which of the available electrodes are used to deliver stimulation signals. The switch module may include a switch array, switch matrix, multiplexer, or any other type of switching device suitable to selectively couple stimulation energy to selected electrodes. In other examples, however, therapy module 94 may independently deliver stimulation to electrodes 40, 42, 44, 46, 48, 50, and 52 or selectively sense via one or more of electrodes 40, 42, 44, 46, 48, 50, and 52 without a switch matrix.

Sensing module 96 monitors signals from at least two of electrodes 40, 42, 44, 46, 48, 50, and 52 in order to monitor electrical activity of heart 14, e.g., via electrocardiogram (ECG) signals. Sensing module 96 may also include a switch module to select the available electrodes 40, 42, 44, 46, 48, 50, and 52 that are used to sense the electrical activity of heart 14. In some examples, processor 90 may select the electrodes that function as sense electrodes via the switch module within sensing module 96, e.g., by providing signals via a data/address bus. In some examples, sensing module 96 includes one or more sensing channels, each of which may comprise an amplifier. In response to the signals from processor 90, the switch module within sensing module 96 may couple the outputs from the selected electrodes to one of the sensing channels.

In some examples, one channel of sensing module 96 may include an R-wave amplifier that receives signals from electrode 42, which may be used for pacing and sensing electrical cardiac activity from tissue proximate to right ventricle 26 (FIG. 2A) of heart 14. Another channel may include another R-wave amplifier that receives signals from electrode 44, which is used for pacing and sensing proximate to left ventricle 28 of heart 14. In some examples, the R-wave amplifiers may take the form of an automatic gain controlled amplifier that provides an adjustable sensing threshold as a function of the measured R-wave amplitude of the heart rhythm.

In addition, in some examples, one channel of sensing module 96 may include a P-wave amplifier that receives signals from electrode 40, which is used for pacing and sensing electrical cardiac activity in tissue proximate to right atrium 30 of heart 14. As an alternative, the P-wave amplifier may receive signals from one or more of electrodes 46, 48, and 50, which may be positioned proximate to right atrium 30 of heart 14. In some examples, the P-wave amplifier may take the form of an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing module 96 may be selectively coupled to housing electrode 52 or other sensing electrodes (not shown) with or instead of one or more of electrodes 40, 42, 44, 46, 48, and 50, e.g., for unipolar sensing of R-waves or P-waves in any of chambers 26, 28, 30, and 32 of heart 14.

In some examples, sensing module 96 includes a channel that comprises an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in memory 92 as an electrogram (EGM). In some examples, the storage of such EGMs in memory 92 may be under the control of a direct memory access circuit. Processor 90 may employ digital signal analysis techniques to characterize the digitized signals stored in memory 92 to detect and classify the patient's heart rhythm from the electrical signals. Processor 90 may detect and classify the heart rhythm of patient 12 by employing any of the numerous signal processing methodologies known in the art.

If cardiac therapy module 102 of IMD 16 is configured to generate and deliver pacing pulses to heart 14, processor 90 may include a pacer timing and control module, which may be embodied as hardware, firmware, software, or any combination thereof. The pacer timing and control module may comprise a dedicated hardware circuit, such as an ASIC, separate from other processor 90 components, such as a microprocessor, or a software module executed by a component of processor 90, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided. When “D” is the third letter in the code, it indicates that the sensed signal is used for triggering a ventricular pace after a P-wave.

Intervals defined by the pacer timing and control module within processor 90 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals from sensing module 96 to blank one or more channels, e.g., amplifiers, for a period during and after cardiac therapy module 102 delivers electrical stimulation to heart 14. The durations of these intervals may be determined by processor 90 in response to stored data in memory 92. The pacer timing and control module of processor 90 may also determine the amplitude of the cardiac pacing pulses.

During pacing, escape interval counters within the pacer timing/control module of processor 90 may be reset upon sensing of R-waves and P-waves. Cardiac therapy module 102 may include pacer output circuits that are coupled, e.g., selectively by a switching module, to any combination of electrodes 40, 42, 44, and 52 appropriate for delivery of a bipolar or unipolar pacing pulse to one of the chambers of heart 14. Processor 90 may reset the escape interval counters upon the generation of pacing pulses by cardiac therapy module 102, and thereby control the basic timing of cardiac pacing functions, including anti-tachyarrhythmia pacing.

The value of the count present in the escape interval counters when reset by sensed R-waves and P-waves may be used by processor 90 to measure the durations of R-R intervals, P-P intervals, P-R intervals and R-P intervals, which are measurements that may be stored in memory 92. Processor 90 may use the count in the interval counters to detect a tachyarrhythmia event, such as ventricular fibrillation event or ventricular tachycardia event. Upon detecting a threshold number of tachyarrhythmia events, processor 90 may identify the presence of a tachyarrhythmia episode, such as a ventricular fibrillation episode, a ventricular tachycardia episode, or a non-sustained tachycardia (NST) episode. Examples of tachyarrhythmia episodes that may qualify for delivery of responsive therapy, e.g., by cardiac therapy module 102 and/or neurostimulation module 104, include a ventricular fibrillation episode or a ventricular tachyarrhythmia episode. In the case of a NST, however, processor 90 may not meet the requirements for triggering a therapeutic response, and, thus, processor 90 may continue normal operation.

In some examples, processor 90 may operate as an interrupt driven device, and is responsive to interrupts from pacer timing and control module, where the interrupts may correspond to the occurrences of sensed P-waves and R-waves and the generation of cardiac pacing pulses. Any necessary mathematical calculations to be performed by processor 90 and any updating of the values or intervals controlled by the pacer timing and control module of processor 90 may take place following such interrupts. A portion of memory 92 may be configured as a plurality of recirculating buffers, capable of holding series of measured intervals, which may be analyzed by processor 90 in response to the occurrence of a pace or sense interrupt to determine whether heart 14 of patient 12 is presently exhibiting atrial or ventricular tachyarrhythmia.

In some examples, an arrhythmia detection method may include any suitable tachyarrhythmia detection algorithms. In one example, processor 90 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND GREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. and U.S. Pat. No. 5,755,736 to Gillberg et al. are incorporated herein by reference in their entireties. However, other arrhythmia detection methodologies may also be employed by processor 90 in other examples.

In the examples described herein, processor 90 may identify the presence of an atrial or ventricular tachyarrhythmia episode by detecting a series of tachyarrhythmia events (e.g., R-R or P-P intervals having a duration less than or equal to a threshold) of an average rate indicative of tachyarrhythmia or an unbroken series of short R-R or P-P intervals. The thresholds for determining the R-R or P-P interval that indicates a tachyarrhythmia event may be stored within memory 92 of IMD 16. In addition, the number of tachyarrhythmia events that are detected to confirm the presence of a tachyarrhythmia episode may be stored as a number of intervals to detect (NID) threshold value in memory 92. In some examples, processor 90 may also identify the presence of the tachyarrhythmia episode by detecting a variable coupling interval between the R-waves of the heart signal. For example, if the differences between the coupling intervals are higher than a given threshold over a predetermined number of successive cycles, processor 90 may determine that the tachyarrhythmia is present.

If processor 90 detects an atrial or ventricular tachyarrhythmia based on signals from sensing module 96, and an anti-tachyarrhythmia pacing regimen is desired, timing intervals for controlling the generation of anti-tachyarrhythmia pacing therapies by cardiac therapy module 102 may be loaded by processor 90 into the pacer timing and control module to control the operation of the escape interval counters therein and to define refractory periods during which detection of R-waves and P-waves is ineffective to restart the escape interval counters.

If IMD 16 is configured to generate and deliver defibrillation pulses to heart 14, cardiac therapy module 102 may include a high voltage charge circuit and a high voltage output circuit. In the event that generation of a cardioversion or defibrillation pulse is required, processor 90 may employ the escape interval counter to control timing of such cardioversion and defibrillation pulses, as well as associated refractory periods. In response to the detection of atrial or ventricular fibrillation or tachyarrhythmia requiring a cardioversion pulse, processor 90 may activate a cardioversion/defibrillation control module, which may, like pacer timing and control module, be a hardware component of processor 90 and/or a firmware or software module executed by one or more hardware components of processor 90. The cardioversion/defibrillation control module may initiate charging of the high voltage capacitors of the high voltage charge circuit of cardiac therapy module 102 under control of a high voltage charging control line.

Processor 90 may monitor the voltage on the high voltage capacitor, e.g., via a voltage charging and potential (VCAP) line. In response to the voltage on the high voltage capacitor reaching a predetermined value set by processor 90, processor 90 may generate a logic signal that terminates charging. Thereafter, timing of the delivery of the defibrillation or cardioversion pulse by cardiac therapy module 102 is controlled by the cardioversion/defibrillation control module of processor 90. Following delivery of the fibrillation or tachycardia therapy, processor 90 may return cardiac therapy module 102 to a cardiac pacing function and await the next successive interrupt due to pacing or the occurrence of a sensed atrial or ventricular depolarization.

Cardiac therapy module 102 may deliver cardioversion or defibrillation pulses with the aid of an output circuit that determines whether a monophasic or biphasic pulse is delivered, whether housing electrode 52 serves as cathode or anode, and which electrodes are involved in delivery of the cardioversion or defibrillation pulses. Such functionality may be provided by one or more switches or a switching module of therapy module 94.

In some examples, sensing module 96 may sense non-cardiac electrical signals, e.g., via electrodes 46, 48, and 50 proximate to spinal cord. The non-cardiac signals may be used to initiate and/or adjust therapy delivery from neurostimulation module 104. Examples of non-cardiac electrical signals may include, for example, signals generated by an activity or motion sensor, such as an accelerometer, which may indicate the activity level or posture of patient 12. As patient 12 moves and changes posture, the position of lead 18 (FIG. 2A) relative to spinal cord 38 may change, which may affect the efficacy of neurostimulation therapy. In some examples, processor 90 may detect the patient's posture or activity level via an electrical signal generated by sensing module 96 or a separate sensing module, which may or may not be mechanically coupled to IMD 16, and select different therapy parameter values based on the patient's posture or activity level. Processor 90 may select different therapy parameter values by switching therapy programs (which may be stored in memory 92 of IMD 16) or by modifying one or more therapy parameter values of a stored therapy program.

As another example, sensing module 96 or a separate sensing module, which may or may not be mechanically coupled to IMD 16, may sense neuroelectrogram signals indicative of neural activity, e.g., proximate to the vagus nerve and/or spinal cord 38 (FIG. 1B) of patient 12. In some examples, processor 90 may select different therapy parameter values based on the level of neural activity indicated by the neuroelectrogram signal. Processor 90 may select different therapy parameter values by switching therapy programs (which may be stored in memory 92 of IMD 16) or by modifying one or more therapy parameter values of a stored therapy program.

Neurostimulation module 104 generates neurostimulation signals, which may be pulses as primarily described herein, or continuous time signals, such as sine waves, for delivery to patient 12 via selected combinations of electrodes 40, 42, 44, 46, 48, 50, 52. In some examples, cardiac signals sensed by sensing module 96 may be used to initiate and/or adjust therapy delivery from neurostimulation module 104.

Telemetry module 98 includes any suitable hardware, firmware, software or any combination thereof for communicating with another device, such as programmer 24 (FIG. 1A). Under the control of processor 90, telemetry module 98 may receive downlink telemetry from and send uplink telemetry to programmer 24 with the aid of an antenna, which may be internal and/or external. Processor 90 may provide the data to be uplinked to programmer 24 and the control signals for the telemetry circuit within telemetry module 98, e.g., via an address/data bus. In some examples, telemetry module 98 may provide received data to processor 90 via a multiplexer.

In some examples, processor 90 may transmit atrial and ventricular heart signals (e.g., ECG or EGM signals) produced sensing module 96 to programmer 24. Programmer 24 may interrogate IMD 16 to receive the heart signals. Processor 90 may store heart signals within memory 92, and retrieve stored heart signals from memory 92. Processor 90 may also generate and store marker codes indicative of different cardiac episodes that sensing module 96 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

The various components of IMD 16 are coupled to power source 100, which may include a rechargeable or non-rechargeable battery or a supercapacitor. A non-rechargeable battery may be selected to last for several years, while a rechargeable battery may be inductively charged from an external device, e.g., on a daily or weekly basis. In some examples, power source 100 may include two power sources such that cardiac module 102 and neurostimulation module 104 are powered by separate power sources. In examples in which cardiac module 102 and neurostimulation module 104 are powered by separate power sources, cardiac module 102 and neurostimulation module 104 may use the same power source when one power source is running low. In other examples, power source 100 may only have one power source shared by both cardiac module 102 and neurostimulation module 104. Example implantable medical devices including more than one power source are described in U.S. Provisional Patent Application Ser. No. 61/110,393 to John Burnes et al. (attorney docket no. P0030860.00/1111-091USP1), which is entitled, “IMPLANTABLE MEDICAL DEVICE INCLUDING TWO POWER SOURCES” and was filed Oct. 31, 2008, the entire content of which is incorporated herein by reference.

In some examples, data generated sensing module 96 and stored in memory 92 may be uploaded to a remote server, from which a clinician or another user may access the data to determine whether a potential sensing integrity issue exists. An example of a remote server includes the CareLink Network, available from Medtronic, Inc. of Minneapolis, Minn. An example system may include an external device, such as a server, and one or more computing devices that are coupled to IMD 16 and programmer 24 via a network.

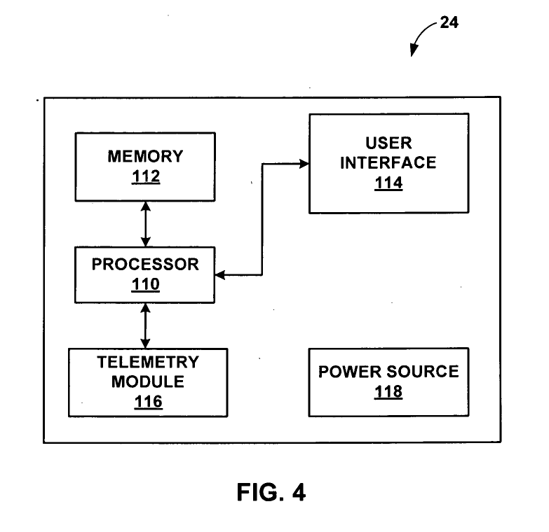

FIG. 4 is block diagram of an example programmer 24. As shown in FIG. 4, programmer 24 includes processor 110, memory 112, user interface 114, telemetry module 116, and power source 118. Programmer 24 may be a dedicated hardware device with dedicated software for programming of IMD 16. Alternatively, programmer 24 may be an off-the-shelf computing device running an application that enables programmer 24 to program IMD 16.

A user may use programmer 24 to select therapy programs (e.g., sets of stimulation parameters), generate new therapy programs, modify therapy programs through individual or global adjustments or transmit the new programs to IMD 16 (FIG. 1A). The therapy programs may be for either or both cardiac therapy module 102 (FIG. 3), neurostimulation module 104 (FIG. 3). The clinician may interact with programmer 24 via user interface 114, which may include display to present graphical user interface to a user, and a keypad or another mechanism for receiving input from a user.

Processor 110 can take the form one or more microprocessors, DSPs, ASICs, FPGAs, programmable logic circuitry, or the like, and the functions attributed to processor 110 herein may be embodied as hardware, firmware, software or any combination thereof. Memory 112 may store instructions that cause processor 110 to provide the functionality ascribed to programmer 24 herein, and information used by processor 110 to provide the functionality ascribed to programmer 24 herein. Memory 112 may include any fixed or removable magnetic, optical, or electrical media, such as RAM, ROM, CD-ROM, hard or floppy magnetic disks, EEPROM, or the like. Memory 112 may also include a removable memory portion that may be used to provide memory updates or increases in memory capacities. A removable memory may also allow patient data to be easily transferred to another computing device, or to be removed before programmer 24 is used to program therapy for another patient. Memory 112 may also store information that controls therapy delivery by IMD 16, such as stimulation parameter values.

Programmer 24 may communicate wirelessly with IMD 16, such as using RF communication or proximal inductive interaction. This wireless communication is possible through the use of telemetry module 116, which may be coupled to an internal antenna or an external antenna. An external antenna that is coupled to programmer 24 may correspond to the programming head that may be placed proximate to the patient's body near the IMD 16 implant site, as described above with reference to FIG. 1A. Telemetry module 116 may be similar to telemetry module 98 of IMD 16 (FIG. 3). As an alternative, IMD 16 ma communicate with programmer 24, another computing device or another implanted medical device via transconductive communication (TCC) utilizing electrodes coupled to IMD 16, e.g., the cardiac and/or neurostimulation electrodes.

Telemetry module 116 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Power source 118 delivers operating power to the components of programmer 24. Power source 118 may include a battery and a power generation circuit to produce the operating power. In some examples, the battery may be rechargeable to allow extended operation. Recharging may be accomplished by electrically coupling power source 118 to a cradle or plug that is connected to an alternating current (AC) outlet. In addition or alternatively, recharging may be accomplished through proximal inductive interaction between an external charger and an inductive charging coil within programmer 24. In other examples, traditional batteries (e.g., nickel cadmium or lithium ion batteries) may be used. In addition, programmer 24 may be directly coupled to an alternating current outlet to power programmer 24. Power source 118 may include circuitry to monitor power remaining within a battery. In this manner, user interface 114 may provide a current battery level indicator or low battery level indicator when the battery needs to be replaced or recharged. In some cases, power source 118 may be capable of estimating the remaining time of operation using the current battery.

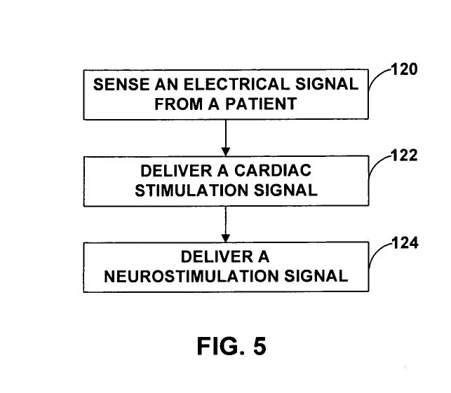

FIG. 5 is a flow diagram of an example technique for delivering both cardiac and neurostimulation therapy to patient 12 via IMD 16 including cardiac therapy module 102 and neurostimulation module 104 (FIG. 3) in a common housing 54 (FIG. 2A). Sensing module 96 (FIG. 3) of IMD 16 may sense an electrical cardiac signal from patient 12 (120). As described in further detail with respect to FIG. 3, sensing module 96 may sense electrical signals attendant to the depolarization and repolarization of heart 14 via electrodes coupled to lead 18. Sensing module 96 may, additionally or alternatively, sense other cardiac or non-cardiac signals, such as signals that indicate patient posture, patient activity level, and/or neural activity. Cardiac therapy module 102 may deliver at least one pacing, cardioversion, or defibrillation therapy to heart 14 of patient 12 via an extravascular electrode, e.g., in response to electrical signals sensed by sensing module 96 (122). For example, cardiac therapy module 102 may deliver a defibrillation pulse to heart 14 in response to a detected fibrillation event sensed by sensing module 96.

Neurostimulation module 104 may also deliver a neurostimulation signal to patient 12, e.g., at a vagus nerve or spinal cord 38 of patient 12 (124). The neurostimulation signal may facilitate delivery of the pacing, cardioversion, or defibrillation therapy. For example, the neurostimulation signal may stimulate the autonomic nervous signal of patient 12 to produce a cardiac benefit, e.g., a change in heart rate, improvement in heart failure status, decrease in arrhythmia, and/or change in blood pressure. The neurostimulation therapy delivered by neurostimulation module 104 may aid in reducing the amount and/or intensity of the therapy cardiac therapy module 102 must deliver to support patient 12.

Although many of the cardiovascular monitoring and analysis techniques described herein are performed by or otherwise controlled by processor 90 of IMD 16, in other examples, another device may perform any part of the techniques described herein.

The techniques described in this disclosure, including those attributed to image IMD 16, programmer 24, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. These and other examples are within the scope of the following claims.

1. A system comprising:
a housing;
at least two extravascular electrodes implanted within a patient;
a cardiac therapy module that delivers at least one of pacing, cardioversion, or defibrillation therapy to the patient via the at least two extravascular electrodes;
a neurostimulation electrode; and
a neurostimulation therapy module that delivers a neurostimulation signal to the patient via the neurostimulation electrode, wherein the cardiac therapy module and the neurostimulation therapy module are enclosed in the housing.

2. The system of claim 1, wherein the neurostimulation signal is configured to modulate an autonomic nervous system of the patient.

3. The system of claim 1, wherein the neurostimulation signal is delivered to at least one of a cardiac fat pad, a baroreceptor, a renal nerve, a vagus nerve, a peripheral nerve or a spinal cord of the patient.

4. The system of claim 1, wherein the neurostimulation signal is configured to at least one of modify a heart rate, improve a heart failure status, prevent an arrhythmia, terminate an arrhythmia, decrease a defibrillation threshold, reduce a post-shock recovery time, reduce a need for post-shock pacing or modify a blood pressure of the patient.

5. The system of claim 1, further comprising a housing electrode on an exterior surface of the housing, wherein the cardiac therapy module delivers the at least one of pacing, cardioversion or defibrillation therapy to the patient via at least one of the at least two extravascular electrodes and the housing electrode.

6. The system of claim 1, wherein the at least two extravascular electrodes each comprise at least one of a subcutaneous electrode or a submuscular electrode.

7. The system of claim 1, wherein the at least two extravascular electrodes each comprise at least one of an epicardial electrode or an intramural electrode.

8. The system of claim 1, further comprising a lead that carries the at least two extravascular electrodes and electrically couples the at least two extravascular electrodes to the cardiac therapy module, wherein the lead carries the neurostimulation electrode and electrically couples the neurostimulation electrode to the neurostimulation therapy module.

9. The system of claim 8, wherein the lead comprises a first lead, the system further comprising:
a second lead that carries the neurostimulation electrode; and
a lead connector that mechanically couples the first and second leads, wherein the neurostimulation electrode is electrically coupled to the neurostimulation therapy module via the first and second leads.

10. The system of claim 9, wherein the housing comprises a connector that electrically couples the first lead to the neurostimulation therapy module and the cardiac therapy module, and wherein the first lead electrically couples the second lead to the neurostimulation therapy module and the cardiac therapy module via the connector.

11. The system of claim 8, wherein the lead comprises a first lead, the system further comprising a second lead that carries the neurostimulation electrode and electrically couples the neurostimulation electrode to the neurostimulation therapy module.

12. The system of claim 1, further comprising a processor that controls the cardiac therapy module and the neurostimulation therapy module.

13. The system of claim 12, further comprising a sensing module that senses a cardiac signal of the patient, wherein the processor controls the cardiac therapy module to generate and deliver the at least one of pacing, cardioversion, or defibrillation therapy based on the electrical signal sensed by the sensing module.

14. The system of claim 1, wherein the neurostimulation therapy module delivers the neurostimulation signal to the patient via the neurostimulation electrode and at least one of the extravascular electrodes.

15. A method comprising:
generating at least one of pacing, cardioversion, or defibrillation therapy with a cardiac therapy module in a housing of a medical device;
delivering the at least one of a pacing, cardioversion, or defibrillation therapy to a patient via at least two extravascular electrodes implanted within a patient;
generating a neurostimulation signal with a neurostimulation therapy module in the housing of the medical device; and
delivering the neurostimulation signal to the patient via a neurostimulation electrode.

16. The method claim 15, wherein delivering the neurostimulation signal comprises delivering the neurostimulation signal to modulate an autonomic nervous system of the patient.

17. The method claim 15, wherein delivering the neurostimulation signal comprises delivering the neurostimulation signal to at least one of a cardiac fat pad, a baroreceptor, a renal nerve, a vagus nerve, a peripheral nerve or a spinal cord of the patient.

18. The method claim 15, wherein delivering the neurostimulation signal comprises delivering the neurostimulation signal to at least one of modify a heart rate, improve a heart failure status, prevent an arrhythmia, terminate an arrhythmia, decrease a defibrillation threshold, reduce a post-shock recovery time, reduce a need for post-shock pacing or modify a blood pressure of the patient.

19. The method of claim 15, wherein delivering the at least one of pacing, cardioversion, or defibrillation therapy to the patient comprises delivering the at least one of pacing, cardioversion, or defibrillation therapy to the at least two extravascular electrodes via at least a first conductor of a lead, and wherein delivering the neurostimulation signal to the patient comprises delivering the neurostimulation signal to the neurostimulation electrode via a second conductor of the lead, wherein the first and second conductors are disposed in a common lead body.

20. The method of claim 15, wherein delivering the at least one of pacing, cardioversion, or defibrillation therapy to the patient comprises delivering the at least one of pacing, cardioversion, or defibrillation therapy to the at least two extravascular electrodes via a first lead, and wherein delivering the neurostimulation signal to the patient comprises delivering the neurostimulation signal to the neurostimulation electrode via a second lead that is separate from the first lead.

21. The method of claim 20, wherein the first and second leads are mechanically coupled to each other via a lead connector.

22. The method of claim 15, further comprising sensing an electrical signal from the patient, wherein delivering the at least one of pacing, cardioversion, or defibrillation comprises delivering the at least one of pacing, cardioversion, or defibrillation therapy based on the electrical signal sensed by the sensing module.

23. The method of claim 22, wherein delivering the neurostimulation signal comprises delivering the neurostimulation signal based on the electrical signal sensed by the sensing module.

24. The method of claim 22, wherein sensing the electrical signal from the patient comprises sensing a response of the patient to the neurostimulation signal.

25. The method of claim 22, wherein the response comprises at least one of a cardiac response or a neural response.

26. The method of claim 15, wherein delivering the neurostimulation signal to the patient comprises delivering the neurostimulation signal to the patient via the neurostimulation electrode and at least one of the extravascular electrodes.

27. A system comprising:
means for generating and delivering at least one of pacing, cardioversion, or defibrillation therapy via at least two extravascular electrodes implanted within a patient;
means for generating and delivering a neurostimulation signal to the patient via a neurostimulation electrode; and
a housing enclosing the means for generating and delivering the at least one of pacing, cardioversion, or defibrillation therapy and means for generating and delivering the neurostimulation signal.

28. The system of claim 27, further comprising means for coupling the at least two extravascular electrodes and the neurostimulation electrode to the means for generating and delivering the at least one of pacing, cardioversion, or defibrillation therapy and the means for generating and delivering the neurostimulation signal, respectively, wherein the means for coupling comprises a single lead body.
